(19) United States
(12) Patent Application Publication
(10) Pub. No.: US 2022/0130542 A1
(43) Pub. Date: Apr. 28, 2022

(54) USING MACHINE LEARNING TO ASSESS MEDICAL INFORMATION BASED ON A SPATIAL CELL ORGANIZATION ANALYSIS

(71) Applicant: The Regents Of The University Of Michigan, Ann Arbor, MI (US)

(72) Inventors: Arvind Rao, Ann Arbor, MI (US); Timothy Frankel, Ann Arbor, MI (US)

(21) Appl. No.: 17/489,441

(22) Filed: Sep. 29, 2021

Related U.S. Application Data
(60) Provisional application No. 63/104,286, filed on Oct. 22, 2020.

(51) Int. Cl. G16H 50/20; G06N 7/00; G16H 20/40

(52) U.S. Cl. CPC G16H 50/20; G16H 20/40; G06N 7/005

(57) ABSTRACT

Systems and methods for using machine learning models to diagnose various diseases or conditions, as well as predict responses to various treatments. According to certain aspects, an electronic device may generate a machine learning model using training data including a set of stained cell images and/or other multi-stream data, as well as indications of any diagnosed diseases and/or whether the patients responded to specific treatments. The electronic device may access additional patient data and input the additional patient into the machine learning model. An output(s) of the machine learning model may indicate a probability(ies) of the additional patient having a disease(s) and/or a probability(ies) of the additional patient responding to a specific treatment(s).

### CROSS-REFERENCE TO RELATED APPLICATIONS

This application claims priority to U.S. Patent Application Ser. No. 63/104,286, filed Oct. 22, 2020, the disclosure of which is hereby incorporated by reference in its entirety.

### FIELD

The present disclosure is directed to using machine learning to assess medical information. More particularly, the present disclosure is directed to platforms and technologies for using machine learning to perform a spatial cell organization modeling and analysis to predict diagnoses and assess the success of medical treatments.

### BACKGROUND

Medical diagnosis is the process of determining which disease or condition explains the symptoms or signs of a person, and usually involves multiple steps including an initial diagnostic assessment and various forms of diagnostic testing. When a patient is diagnosed, that patient usually undergoes appropriate treatment that is recommended and/or supervised by one or more physicians, with the hope that the treatment is successful and the patient recovers from the underlying disease or condition.

However, diagnostic error still occurs when a physician diagnoses the wrong disease or condition, or otherwise does not diagnose the underlying disease or condition. Diagnostic error may happen for a variety of reasons, including lack of training, lack of proper testing, and/or patient symptoms being similar to other medical conditions, among other reasons. Diagnostic tests often rely on subjective interpretation of data which can suffer from high incidence of inter-observer variability. Additionally, even in cases in which a patient is correctly diagnosed, there may be multiple treatment options for the diagnosed condition, however the patient may or may not respond positively to one or more of the treatment options, which in certain situations is difficult if not impossible to know prior to administering the one or more treatment options.

Accordingly, there is an opportunity to employ machine learning systems and techniques to efficiently and accurately diagnose certain diseases or conditions, augment the clinical decision making process, as well as to efficiently and accurately predict whether a given patient will respond positively to a specific treatment.

### SUMMARY

In an embodiment, a computer-implemented method of using machine learning to assess medical information is provided. The computer-implemented method may include: training, by a computer processor, a machine learning model using a set of patient data associated with a plurality of patients, the set of patient data (i) generated using a spatial organization modeling technique, and (ii) identifying, for each patient of the plurality of patients, a diagnosed disease of a plurality of diseases; storing the machine learning model in memory; analyzing, by the computer processor using the machine learning model, an additional set of patient data associated with an additional patient, the additional set of patient data generated using the spatial organization modeling technique; and based on the analyzing, outputting, by the machine learning model, a plurality of probabilities of the additional patient having the plurality of diseases, respectively.

In another embodiment, a system for using machine learning to assess medical information is provided. The system may include a processor, a memory storing data associated with a machine learning model, and a non-transitory computer-readable memory interfaced with the processor and the memory. The non-transitory computer-readable memory may store instructions thereon that, when executed by the processor, cause the processor to: train the machine learning model using a set of patient data associated with a plurality of patients, the set of patient data (i) generated using a spatial organization modeling technique, and (ii) identifying, for each patient of the plurality of patients, a diagnosed disease of a plurality of diseases, analyze, by the computer processor using the machine learning model, an additional set of patient data associated with an additional patient, the additional set of patient data generated using the spatial organization modeling technique, and based on the analyzing, output, by the machine learning model, a plurality of probabilities of the additional patient having the plurality of diseases, respectively.

Further, in an embodiment, a computer-implemented method of using machine learning to predict patient response to a treatment for a disease is provided. The computer-implemented method may include: training, by a computer processor, a machine learning model using a set of patient data associated with a plurality of patients each being diagnosed with the disease, the set of patient data (i) generated using a spatial organization modeling technique, and (ii) indicating, for each patient of the plurality of patients, whether that patient responded to the treatment; storing the machine learning model in memory; analyzing, by the computer processor using the machine learning model, an additional set of patient data associated with an additional patient, the additional set of patient data generated using the spatial organization modeling technique; and based on the analyzing, outputting, by the machine learning model, a probability of whether the additional patient will respond to the treatment.

### BRIEF DESCRIPTION OF THE FIGURES

FIG. 1A depicts an overview of components and entities associated with the systems and methods, in accordance with some embodiments.

FIG. 1B depicts an overview of certain components configured to facilitate the systems and methods, in accordance with some embodiments.

FIG. 2 depicts an representations of various techniques and analyses in association with using a machine learning model to perform a spatial cell organization analysis to assess medical information.

FIG. 3 depicts an representations of various techniques and analyses in association with training a machine learning model to be used to perform a spatial cell analysis to assess medical information.

FIG. 4 is an example flowchart associated with using machine learning to assess medical information, in accordance with some embodiments.

### DETAILED DESCRIPTION

The present embodiments may relate to, inter alia, platforms and technologies for using machine learning to assess medical information. According to certain aspects, a computing device may train a machine learning model using various information derived from patient data, such as a set of images resulting from cell staining, lab values, genetics, and/or the like, where the patient data indicates a diagnosed disease or condition of the associated patients. The computing device may perform a spatial organization modeling technique (e.g., geographically weighted regression (GWR)) based on the locations of various types of cells depicted in the set of images, where the computing device may use the resulting data to train the machine learning model. Additionally, the computing device may input additional patient data associated with an additional patient into the trained machine learning model, which may output a set of probabilities of the additional patient having a respective set of diseases or conditions. Additionally, the computing device may use the machine learning model to predict whether patients will respond to certain treatments.

The systems and methods therefore offer numerous benefits. In particular, the use of various machine learning techniques enable the systems and methods to accurately, more consistently, and dynamically diagnose patients with certain diseases or conditions. This is particularly beneficial in situations in which conventional diagnoses consume a lot of time and effort, such as where diagnosis is based on subjective interpretation of data with high degrees of inter-observer variability and/or are otherwise erroneous. Additionally, the use of the various machine learning techniques enable the systems and methods to accurately, more consistently, and dynamically determine a probability of whether a particular patient will respond to certain treatments to treat diagnosed diseases or conditions. Thus, the systems and methods will lead to the preparation of more accurate and effective treatment plans, which will lead to the increased chances of patients recovering from illness. These benefits decrease the strain on healthcare systems and reduce the costs incurred by insured individuals and other individuals. This benefit will be particularly experienced by individuals who live remotely from centers with expertise in subjective interpretation of diagnostic data. It should be appreciated that additional benefits are envisioned.

The systems and methods discussed herein address a challenge that is particular to patient care. In particular, the challenge relates to a difficulty in accurately and effectively diagnosing patient illness, as well as a difficulty in accurately and effectively predicting whether patients will respond positively to certain treatments. In existing situations, physicians may misdiagnose certain conditions, which may result in patients being treated incorrectly or inefficiently. Additionally, patients may undertake a certain treatment that is intended to help the patient but that the patient does not respond to (and that may actually harm the patient). The systems and methods offer improved capabilities to solve these problems by using a trained machine learning model to accurately diagnose diseases and conditions, as well as predict whether patients will positively respond to certain treatments. Further, because the systems and methods employ communication between and among multiple devices, the systems and methods are necessarily rooted in computer technology in order to overcome the noted shortcomings that specifically arise in the realm of patient care.

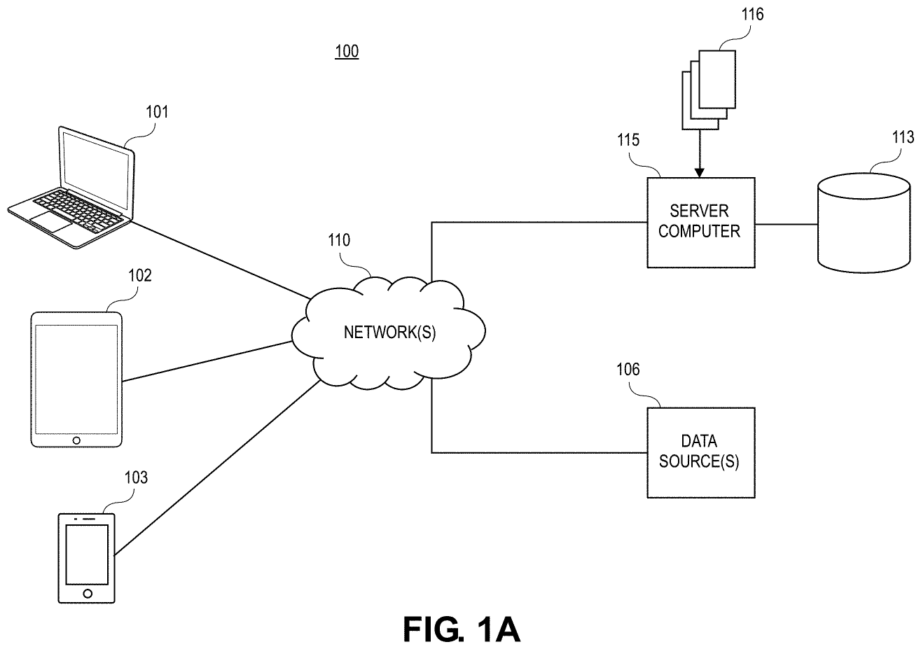

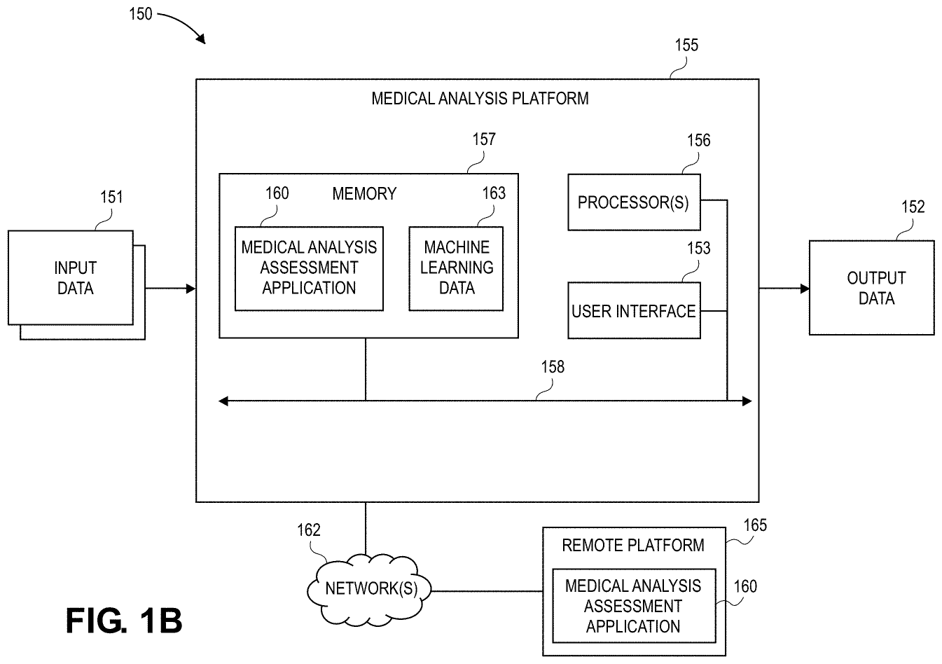

FIG. 1A illustrates an overview of a system 100 of components configured to facilitate the systems and methods. It should be appreciated that the system 100 is merely an example and that alternative or additional components are envisioned.

As illustrated in FIG. 1A, the system 100 may include a set of electronic devices 101, 102, 103. Each of the electronic devices 101, 102, 103 may be any type of electronic device such as a digital microscope, mobile device (e.g., a smartphone), desktop computer, notebook computer, tablet, phablet, GPS (Global Positioning System) or GPS-enabled device, smart watch, smart glasses, smart bracelet, wearable electronic, PDA (personal digital assistant), pager, computing device configured for wireless communication, and/or the like. In embodiments, any of the electronic devices 101, 102, 103 may be an electronic device associated with an individual (e.g., a patient) or an entity such as a company, business, corporation, or the like (e.g., a server computer or machine).

The electronic devices 101, 102, 103 may communicate with a server computer 115 via one or more networks 110. In embodiments, the network(s) 110 may support any type of data communication via any standard or technology (e.g., GSM, CDMA, VoIP, TDMA, WCDMA, LTE, EDGE, OFDM, GPRS, EV-DO, UWB, Internet, IEEE 802 including Ethernet, WiMAX, Wi-Fi, Bluetooth, 4G/5G/6G, Edge, and others). The server computer 115 may be associated with an entity such as a company, business, corporation, or the like. In some embodiments, the server computer 115 may be associated with medical care, such as a pharmacy, hospital, medical practice, physician, pharmaceutical company, or the like. The server computer 115 may include various components that support communication with the electronic devices 101, 102, 103.

The server computer 115 may communicate with one or more data sources 106 via the network(s) 110. In embodiments, the data source(s) 106 may compile, store, or otherwise access information associated the patient care, including the diagnosis and treatment of various conditions or diseases. For example, the data source 106 may be a hospital or university medical center that researches diseases and the treatments thereof. It should be appreciated that alternative and additional data sources are envisioned.

Generally, the data source(s) 106 may store information indicative of patient tissue samples and existing patient diagnoses. In particular, the information may include a set of images resulting from cell staining from the tissue samples, as well as indications of which patients have which diagnosed conditions, and/or information indicating or identifying various genetics/genomics, mutations, proteomics, lab values, and clinical measurements. For example, the information may include a first set of stained images for a first patient and an indication that the first patient has been diagnosed with chronic pancreatitis (CP), and a second set of stained images for a second patient and an indication that the second patient has been diagnosed with intraductal papillary mucinous neoplasms (IPMN). In embodiments, the information may indicate which of the patients responded to certain treatments. For example, the tissue samples may be of patients with lung cancer, and the information may indicate which of the patients did (or did not) respond to immunotherapy treatment. The server computer may analyze this data according to the functionalities as described herein, which may result in a set of training datasets 116. In some implementations, the server computer 115 may access the raw data or information (and/or training dataset 116) from one or more of the electronic devices 101, 102, 103.

The server computer 115 may receive, access, or generate the training dataset 116, and may employ various machine learning techniques, calculations, algorithms, and the like to generate a set of machine learning models using the training dataset 116. In particular, the server computer 115 may initially train a set of machine learning models using the training dataset 116 and then apply or input a validation set into a set of generated machine learning models to determine which of the machine learning models is most accurate or otherwise may be used as the final or selected machine learning model.

According to embodiments, the server computer 115 may input, into the generated machine learning model, a set of input data (which may be a set of real-world patient data) associated with an additional set of patients, a result or output of which may include a set of probabilities of each patient of the additional set of patients having the respective diagnosed conditions. In embodiments, the result or output may include a set of probabilities of the patients responding to a particular treatment(s). A user of the electronic devices 101, 102, 103 (e.g., a physician) may review the result(s) or output(s) and advise on treatments, diagnoses, and/or the like related to the subject condition. In embodiments, a user may access the result(s) or output(s) directly from the server computer 115.

The server computer 115 may be configured to interface with or support a memory or storage 113 capable of storing various data, such as in one or more databases or other forms of storage. According to embodiments, the storage 113 may store data or information associated with the machine learning models that are generated by the server computer 115. Additionally, the server computer 115 may access the data associated with the stored machine learning models to input a set of inputs into the machine learning models.

Although depicted as a single server computer 115 in FIG. 1A, it should be appreciated that the server computer 115 may be in the form of a distributed cluster of computers, servers, machines, cloud-based services, or the like. In this implementation, the entity may utilize the distributed server computer(s) 115 as part of an on-demand cloud computing platform. Accordingly, when the electronic devices 101, 102, 103 interface with the server computer 115, the electronic devices 101, 102, 103 may actually interface with one or more of a number of distributed computers, servers, machines, or the like, to facilitate the described functionalities.

Although three (3) electronic devices 101, 102, 103, and one (1) server computer 115 are depicted in FIG. 1A, it should be appreciated that greater or fewer amounts are envisioned. For example, there may be multiple server computers, each one associated with a different entity. FIG. 1B depicts more specific components associated with the systems and methods.

FIG. 1B an example environment 150 in which a set of input data 151 is processed into output data 152 via a medical analysis platform 155, according to embodiments. In one implementation, the set of input data 151 may be a training dataset. The medical analysis platform 155 may be implemented on any computing device, including the server computer 115 (or in some implementations, one or more of the electronic devices 101, 102, 103) as discussed with respect to FIG. 1A. Components of the computing device may include, but are not limited to, a processing unit (e.g., processor(s) 156), a system memory (e.g., memory 157), and a system bus 158 that couples various system components including the memory 157 to the processor(s) 156. The computing device may further include various communication components (e.g., transceivers and ports) that may facilitate data communication with one or more additional computing devices.

In some embodiments, the processor(s) 156 may include one or more parallel processing units capable of processing data in parallel with one another. The system bus 158 may be any of several types of bus structures including a memory bus or memory controller, a peripheral bus, or a local bus, and may use any suitable bus architecture. By way of example, and not limitation, such architectures include the Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus (also known as Mezzanine bus).

The medical analysis platform 155 may further include a user interface 153 configured to present content (e.g., the content of the input data 151 and/or the output data 152, and information associated therewith). Additionally, a user may make selections to the content via the user interface 153, such as to navigate through different information, review certain input data, and/or other actions. The user interface 153 may be embodied as part of a touchscreen configured to sense touch interactions and gestures by the user. Although not shown, other system components communicatively coupled to the system bus 158 may include input devices such as cursor control device (e.g., a mouse, trackball, touch pad, etc.) and keyboard (not shown). A monitor or other type of display device may also be connected to the system bus 158 via an interface, such as a video interface. In addition to the monitor, computers may also include other peripheral output devices such as a printer, which may be connected through an output peripheral interface (not shown).

The memory 157 may include a variety of computer-readable media. Computer-readable media may be any available media that can be accessed by the computing device and may include both volatile and nonvolatile media, and both removable and non-removable media. By way of non-limiting example, computer-readable media may comprise computer storage media, which may include volatile and nonvolatile, removable and non-removable media implemented in any method or technology for storage of information such as computer-readable instructions, routines, applications (e.g., a medical analysis assessment application 160), data structures, program modules or other data.

Computer storage media may include, but is not limited to, RAM, ROM, EEPROM, FLASH memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by the processor 156 of the computing device.

The medical analysis platform 155 may operate in a networked environment and communicate with one or more remote platforms, such as a remote platform 165, via a network(s) 162, such as a local area network (LAN), a wide area network (WAN), telecommunications network, or other suitable network. The remote platform 165 may be implemented on any computing device, including one or more of the electronic devices 101, 102, 103, or the server computer 115 as discussed with respect to FIG. 1A, and may include many or all of the elements described above with respect to the platform 155. In some embodiments, as will be described herein, the medical analysis assessment application 160 as will be further described herein may be stored and executed by the remote platform 165 instead of by or in addition to the platform 155.

The medical analysis assessment application 160 may employ machine learning techniques such as, for example, a regression analysis (e.g., a logistic regression, linear regression, random forest regression, probit regression, or polynomial regression), classification analysis, k-nearest neighbors, decisions trees, random forests, boosting, neural networks, support vector machines, deep learning, reinforcement learning, Bayesian networks, or the like. When the data 151 is a training dataset (which may include real-world patient data), the medical analysis assessment application 160 may analyze/process the data 151 to generate the machine learning model for storage as part of machine learning data 163 that may be stored in the memory 157.

When the data 151 comprises real-world patient data to be analyzed using the machine learning model, the medical analysis assessment application 160 may analyze or process the data 151 using the machine learning model to generate the output data 152 that may comprise various metrics and information corresponding to the trained machine learning model. In one scenario, if the machine learning model is trained to determine patient diagnoses, the output data 152 may include a set of probabilities for a patient having a respective set of diseases or conditions. In another scenario, if the machine learning model is trained to determine treatment responsiveness, the output data 152 may include a proability(ies) of a patient responding to a particular treatment(s).

Generally, each of the data 151 and the data 152 may be embodied as any type of electronic document, file, template, etc., that may include various textual content, and may be stored in memory as program data in a hard disk drive, magnetic disk and/or optical disk drive in the medical analysis platform 155 and/or the remote platform 165.

The medical analysis assessment application 160 (or another component) may cause the output data 152 (and, in some cases, the training or input data 151) to be displayed on the user interface 153 for review by the user of the medical analysis platform 155. The user may select to review and/or modify the displayed data. For instance, the user may review the output data 152 to assess the patient diagnoses and/or a predicted probability of treatment responsiveness. According to embodiments, some or all of the output data 152 may be added to the machine learning model to effectively update the machine learning model, which may be used in subsequent analyses of input data.

In general, a computer program product in accordance with an embodiment may include a computer usable storage medium (e.g., standard random access memory (RAM), an optical disc, a universal serial bus (USB) drive, or the like) having computer-readable program code embodied therein, wherein the computer-readable program code may be adapted to be executed by the processor 156 (e.g., working in connection with an operating systems) to facilitate the functions as described herein. In this regard, the program code may be implemented in any desired language, and may be implemented as machine code, assembly code, byte code, interpretable source code or the like (e.g., via Golang, Python, Scala, C, C++, Java, Actionscript, Objective-C, Javascript, CSS, XML, SAS, R, Stata, AI libraries). In some embodiments, the computer program product may be part of a cloud network of resources.

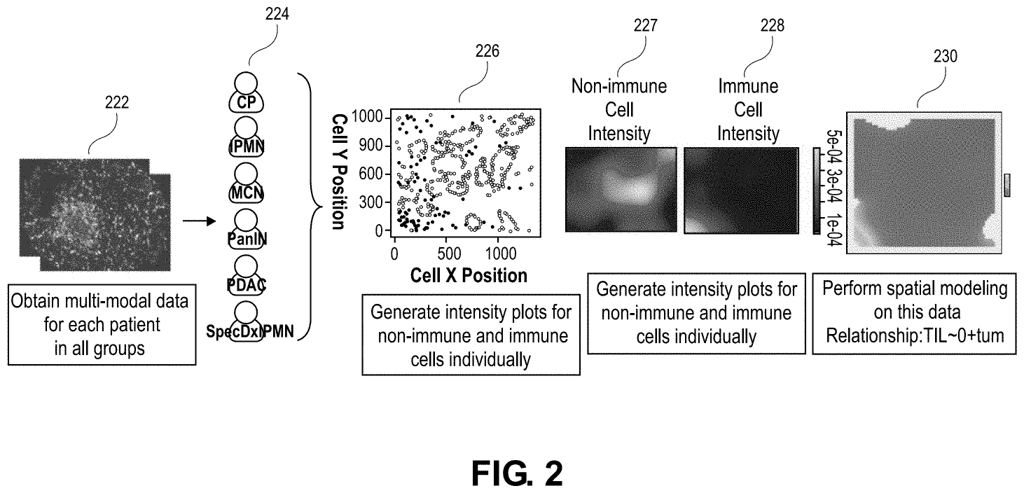

FIG. 2 depicts representations of various calculations that may be performed by a server computer (such as the server computer 115 as discussed with respect to FIG. 1A). It should be appreciated that alternative or additional components are envisioned

As indicated by 222, the server may initially obtain a set of stained images corresponding to a set of tissue samples of a set of patients (i.e., resulting from a cell staining procedure), where each patient may have one or more stained images in the set of stained images. In embodiments, each of the set of stained images may indicate a location (e.g., x and y coordinates) of various types of cells within that stained image. The embodiments herein discuss two different types of cells that may be illustrated in each of the set of stained images: non-immune cells (which may be malignant (i.e., tumor cells) or benign (i.e., epithelial cells or fibroblasts)) and immune cells (i.e., t-cells). However, it should be appreciated that many different types of cells may be illustrated in the set of stained images. Additionally, the set of stained images may also include non-cellular components of the microenvironment such collagen fibers and mucin, among other components.

In addition to the set of stained images, the server may also receive or access information indicative of a diagnosed condition, illness, or disease (generally, as used herein, “disease”). That is, each of the set of stained images may have associated a diagnosed disease for the particular patient associated with that stained image. The embodiments herein discuss the following diagnosed diseases (as indicated in 224 of FIG. 2): chronic pancreatitis (CP), intraductal papillary mucinous neoplasms (IPMN), mucinous cystic neoplasms (MCN), pancreatic intraepithelial neoplasia (PanIN), pancreatic ductal adenocarcinoma (PDAC), and Special Dx IPMN. However, it should be appreciated that other diagnosed diseases for the patients are envisioned.

The server may be additionally configured to access or generate a set of coordinates (e.g., x and y coordinates) of the phenotype (i.e., type) of each cell in each of the set of stained images. In particular, the server may generate, for each of the set of stained images, a plot or visual indicating the locations and type of the cells in that stained image. FIG. 2 illustrates an example visual 226 corresponding to one of the set of stained images 222, where the non-immune cells and the immune cells may be plotted and color-coded (e.g., red for non-immune cells and blue for immune cells).

Further, the server may be configured to generate, for each of the visuals corresponding to the set of stained images 222, a set of intensity plots indicating the presence (and absence) of the corresponding cell type as included in the corresponding visual generated from the corresponding stained image. In embodiments, the server may generate an intensity plot for each type of cell included in the corresponding visual. For example, FIG. 2 illustrates an intensity plot 227 indicating the presence of the non-immune cells included in the visual 226, and an intensity plot 228 indicating the presence of the immune cells included in the visual 226. The intensity plot 227 indicates that the non-immune cells are mainly concentrated in the middle and the top right corner, and the intensity plot 228 indicates that the immune cells are mainly concentrated in the bottom left corner. It should be appreciated that the server may generate the intensity plots using any technique, algorithm, calculation, or the like such as, for example, image marker intensities, spatial density modeling, local averaging, and/or the like.

The server may be further configured to perform a spatial organization modeling technique on the generated or accessed data. The embodiments discussed herein describe the spatial organization modeling technique as a GWR technique, however it should be appreciated that alternative or additional techniques are envisioned.

In embodiments, the server may be configured to perform a GWR analysis on the set of intensity plots for a corresponding visual (i.e., the GWR analysis may be performed on the combination or the full set of intensity plots that are generated from a single corresponding visual). Generally, GWR is a modeling technique and visualization tool to explore patterns of spatial data, and enables assessment of the spatial heterogeneity in the estimated relationships between the corresponding independent and dependent variables.

In the example illustrated in FIG. 2, the server may generate a GWR output 230 based on performing the GWR analysis on the intensity plots 227, 228, where the data in the intensity plot 227 represents the independent variable (i.e., the spatial presence of non-immune cells) and the data in the intensity plot 228 represents the dependent variable (i.e., the spatial presence of immune cells), or vice-versa. Therefore, the GWR output 230 expresses the intensity plot 227 in terms of the intensity plot 228). The resulting GWR output 230 is a set of values representative of the regression coefficient associated with the dependent and independent variables, where the lower the value of the regression coefficient in a particular location, the lower the spatial overlap/relationship between the dependent and independent variables (and the less intense the signal color), and the higher the value of the regression coefficient in a particular location, the higher the spatial overlap/relationship between the dependent and independent variables (and the more intense the signal color). Because a single patient may have multiple stained images in the set of stained images 222, the server may generate multiple respective GWR outputs for that single patient.

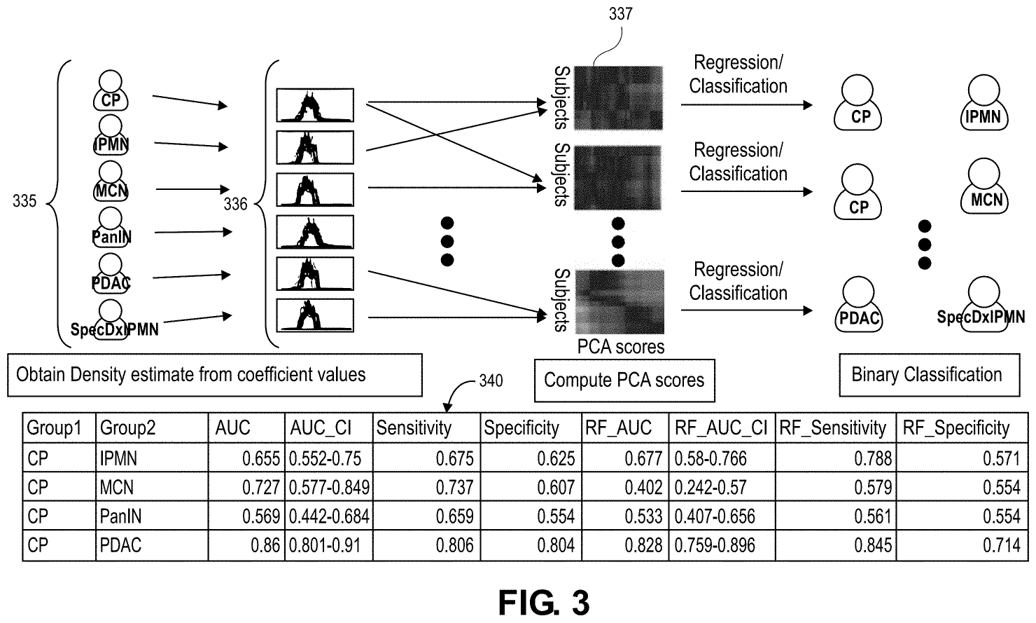

According to embodiments, the set of GWR outputs (including the GWR output 230) generated by the server may be used to train a machine learning model(s). FIG. 3 illustrates representations of various steps and calculations that the server may undertake to train the machine learning model(s).

Initially, the server may generate, for each GWR output in the set of GWR outputs, a two-dimensional curve or density estimate that represents the values included in the respective GWR output, where each two-dimensional curve or density estimate is also associated with or indicates a diagnosed disease. FIG. 3 indicates a set of diseases 335 (as shown: CP, IPMN, MCN, PanIN, PDAC, and SpecDXIPMN), and respective sets of curves 336. Each set of curves in the sets of curves 336 corresponds to one of the set of diseases 335, where the set of curves includes the two-dimensional curves corresponding to the GWR outputs for patients each having the corresponding disease. For example, the set of curves corresponding to CP includes a plurality of individual curves respectively corresponding to the GWR outputs for a plurality of patients each having CP.

The server may perform a data transformation (e.g., a principal component analysis (PCA)) between any two of the sets of curves 336 to analyze similarities and differences between the two sets of curves. In performing the PCA, the server may generate a set of data vectors indicative of the similarities and differences between the two sets of curves. As shown in FIG. 3, a representation 337 depicts PCA scores (i.e., data vectors) that result from performing a PCA analysis on the set of curves corresponding to CP and the set of curves corresponding to IPMN. The server may perform the comparison between the sets of curves in a “round-robin” manner (i.e., each set of curves for each disease is compared to every other set of curves for the remaining disease).

In comparing the sets of curves, the server may employ various techniques, calculations, models, and/or the like, to assess certain metrics, values, or parameters (generally, “metrics”) indicative of the comparison. In one embodiment, the server may employ a probit model or regression. In another embodiment, the server may employ a regression (e.g., a random forest (RF) regression) or classification model. More generally, any type of model within predictive model class can be used in this part. According to embodiments, the metrics may include an area under curve (AUC), an AUC_CI (i.e., confidence interval), an accuracy, a sensitivity, and a specificity.

FIG. 3 illustrates a table 340 indicating the metrics resulting from the comparisons between the sets of curves. In the table 340, the columns that include the text “RF” represent metrics resulting from an RF regression/classification, and the columns that lack the text “RF” represent metrics resulting from a probit regression. Generally, the closer the AUC value is to 1.00 for a particular comparison, the larger the difference(s) between the classes corresponding to the compared curves, relative to random classification (e.g., a coin toss) that were compared (and the more accurately the disease may be diagnosed); and the closer the AUC value is to 0.50 for a particular comparison, the smaller the difference(s) between the sets of curves that were compared (and the less accurately the disease may be diagnosed), like a random classification. For example, row 4 of the table 340 indicates an AUC of 0.86 for the discrimination of the two classes corresponding to CP and PDAC and row 3 of the table 340 indicates an AUC of 0.569 for the discrimination of the two classes corresponding to CP and PanIN, which indicates that there are more differences between CP and PDAC (and are thus diagnosed more accurately) than between CP and PanIN (and are thus diagnosed with less accuracy).

By generating and compiling the sets of curves 336 and performing the analyses on the sets of curves 336, the server may train a machine learning model that may be used to diagnose (or predict a diagnosis of) a disease that a patient may have. In operation, a set of stained images may be obtained or accessed for a particular patient who may not have a diagnosed disease, where the objective may be to analyze the set of stained images using the trained machine learning model to calculate a probability(ies) that the particular patient has a particular disease(s). It should be appreciated that the server may perform the same analyses for patients who already have a diagnosis for a particular disease (e.g., as a second opinion).

Initially, the server may analyze the set of stained images for the particular patient to identify or generate a set(s) of x-y coordinates representative of the cell phenotypes depicted in the set of stained images (e.g., as referenced by 226 in FIG. 2). From each of the set of x-y coordinates, the server may generate a set of intensity plots, where each of the intensity plots may correspond to a different cell type as identified in the x-y coordinates (e.g., as referenced by 228 in FIG. 2). Next, the server may generate, from the set of intensity plots in combination, a GWR output (e.g., as referenced in 230 in FIG. 2) representing the spatial overlap, association, or relationship between and among the cells indicated in the set of intensity plots.

The server may then input the generated GWR output(s) into the trained machine learning model for analysis. According to embodiments, the trained machine learning model may output a set of probabilities respectively associated with a set of diseases on which the machine learning model was trained. For example, if the machine learning model was trained to diagnose CP, IPMN, MCN, PanIN, PDAC, and SpecDxIPMN, the machine learning model may output a set of probabilities for the particular patient respectively having CP, IPMN, MCN, PanIN, PDAC, or SpecDxIPMN. In embodiments, the set of probabilities may total 100%. Continuing with the example, the machine learning model may output the following set of probabilities: 70% for CP, 15% for IPMN, 10% for MCN, 2% for PanIN, 2% for PDAC, and 1% for PsecDxIPMN.

The server may continuously update the trained machine learning model based on any generated output(s) and/or additional sets of training data. In this way, the machine learning model may reflect the analyses of up-to-date data.

According to embodiments, the systems and methods discussed herein may be applied in additional contexts and for additional applications. In one scenario, the systems and methods may be used to predict responses to various treatments for certain diseases. The systems and methods and described herein discuss the treatment as immunotherapy to treat certain diseases, however it should be appreciated that the systems and methods may be used to predict responses to alternative or additional treatments for various diseases such as, for example, responses to chemotherapy or steroids for inflammatory bowel disease, and/or others.

Generally, immunotherapy is a treatment of a condition or disease (e.g., various types of cancer, various types of autoimmune conditions, and others) by activating or suppressing the immune system, where immunotherapies designed to elicit or amplify an immune response are classified as activation immunotherapies, while immunotherapies that reduce or suppress an immune response are classified as suppression immunotherapies.

A consideration of immunotherapy is that a certain percentage of patients do not respond to immunotherapy to treat an underlying condition or disease. For example, studies show that a certain percentage of patients having lung cancer do not respond to immunotherapy treatment. This can be harmful to these patients because the treatment itself may exacerbate underlying autoimmune conditions, without the benefit of the intended immunotherapy response. Accordingly, it would be beneficial to be able to more accurately predict, in advance of administering immunotherapy treatments, which patients are likely to respond (or not respond) to immunotherapy.

According to embodiments, the systems and methods as discussed herein may be used to predict a patient's response to immunotherapy. In operation, a machine learning model may be trained using a set of training data that includes a set of stained images corresponding to a set of patients, where the set of stained images include different cell phenotypes that may be mapped as coordinates using the techniques as discussed herein. Additionally, the set of training data indicates a disease or condition had by the patient (e.g., lung cancer) as well as an indication of whether the patient responded to immunotherapy (e.g., as a Boolean or a continuous value). The machine learning model may thus generally indicate which spatial cell patterns may be indicative of whether the patients will respond to immunotherapy treatment.

A set of patient data associated with a set of patients who may not yet have been treated with immunotherapy may be input into the machine learning model. The set of patient data may be analyzed using the machine learning model, in accordance with the analyses as discussed herein, and the machine learning model may output, for each patient in the set of patients, a probability of whether that patient will respond to immunotherapy treatment. In this regard, a physician may be able to more effectively determine to recommend immunotherapy to his or her patients.

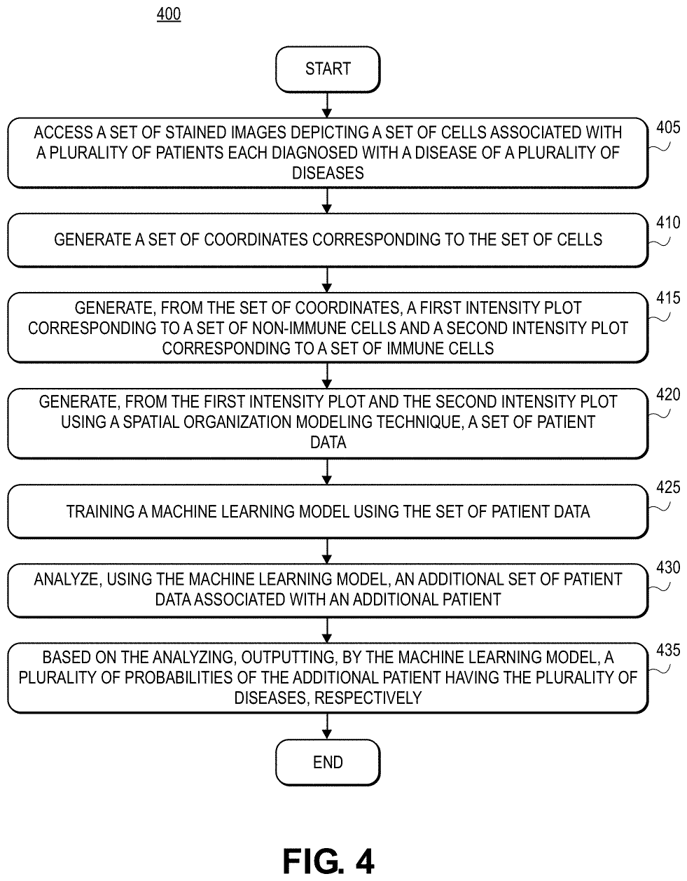

FIG. 4 depicts is a block diagram of an example method 400 for using machine learning to assess medical information. The method 400 may be facilitated by an electronic device (such as the server computer 115 or components associated with the medical analysis platform 155 as discussed with respect to FIGS. 1A and 1B) that may be in communication with additional devices or data sources.

The method 400 may begin when the electronic device accesses (block 405) a set of stained images depicting a set of cells associated with a plurality of patients each diagnosed with a disease of a plurality of diseases. In embodiments, a given patient of the plurality of patients may have associated one or more of the set of stained images, and the set of cells may include a set of immune cells and a set of non-immune cells (and/or other types of cells). The electronic device may alternatively or additional access general patient data, such as genetics/genomics, proteomics, mutations, lab values, and clinical measurements.

The electronic device may generate (block 410) a set of coordinates corresponding to the set of cells. In embodiments, the electronic device may identify a set of locations of the set of cells with the set of stained images, and may generate the set of coordinates as x-y coordinates based on the set of locations, where the set of coordinates may delineate the corresponding types of cells depicted in the set of stained images. The electronic device may generate (block 415), from the set of coordinates, a first intensity plot corresponding to the set of non-immune cells and a second intensity plot corresponding to the set of immune cells. In embodiments, the intensity plots may depict the presence of the respective sets of cells at various locations corresponding to the set of coordinates.

The electronic device may generate (block 420), from the first intensity plot and the second intensity plot using a spatial organization modeling technique, a set of patient data. In embodiments, generating the set of patient data may include, for each diagnosed disease of the plurality of diseases using a set of coefficient values generated using the GWR, generating a set of density estimate curves.

The electronic device may train (block 425) a machine learning model using the set of patient data. In embodiments in which the electronic device generates the set of density estimate curves, the electronic device may, for each pair of diagnosed diseases in the plurality of diseases, generate a set of statistical measures between a first one of the pair of diagnosed disease and a second one of the pair of diagnosed diseases, where the set of statistical measures may be indicative of a set of differences between a first set of density estimate curves corresponding to the first one of the pair of diagnosed diseases and a second set of density estimate curves corresponding to the second one of the pair of diagnosed diseases. Additionally, the electronic device may generate the set of statistical measures using a regression or classification, such as a probit regression or a random forest (RF) regression.

The electronic device may analyze (block 430), using the machine learning model, an additional set of patient data associated with an additional patient. In embodiments, the electronic device may generate the additional set of patient data using the same techniques and analyses as described with respect to blocks 405, 410, 415, and 420.

Based on the analysis of block 430, the electronic device may output (block 435), from the machine learning model, a plurality of probabilities of the additional patient having the plurality of diseases, respectively. In embodiments, the sum of the plurality of probabilities may equal 100%.

Alternatively or additionally, the electronic device may execute or facilitate various steps of the method 400 to predict patient response to certain disease treatments (e.g., immunotherapy). In these embodiments, the electronic device may train the corresponding machine learning model using a set of patient data corresponding to a plurality of patients, where the set of patient data is generated using a GWR technique and also indicates, for each patient of the plurality of patients, whether that patient responded to a subject treatment.

Additionally, the electronic device may analyzing, using the machine learning model, an additional set of patient data associated with an additional patient, and output, by the machine learning model, a probability of whether the additional patient will respond to the subject treatment.

Although the following text sets forth a detailed description of numerous different embodiments, it should be understood that the legal scope of the invention may be defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a non-transitory, machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that may be permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that may be temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules may provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it may be communicatively coupled. A further hardware module may then, at a later time, access the memory device to retrieve and process the stored output. Hardware modules may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment, or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment may be included in at least one embodiment. The appearances of the phrase “in one embodiment” in various places in the specification are not necessarily all referring to the same embodiment.

As used herein, the terms “comprises,” “comprising,” “may include,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also may include the plural unless it is obvious that it is meant otherwise.

This detailed description is to be construed as examples and does not describe every possible embodiment, as describing every possible embodiment would be impractical.

What is claimed is:

1. A computer-implemented method of using machine learning to assess medical information, the computer-implemented method comprising:
training, by a computer processor, a machine learning model using a set of patient data associated with a plurality of patients, the set of patient data (i) generated using a spatial organization modeling technique, and (ii) identifying, for each patient of the plurality of patients, a diagnosed disease of a plurality of diseases;
storing the machine learning model in memory;
analyzing, by the computer processor using the machine learning model, an additional set of patient data associated with an additional patient, the additional set of patient data generated using the spatial organization modeling technique; and
based on the analyzing, outputting, by the machine learning model, a plurality of probabilities of the additional patient having the plurality of diseases, respectively.

2. The computer-implemented method of claim 1, further comprising:
accessing a set of stained images depicting a set of cells associated with the additional patient, the set of cells comprising a set of non-immune cells and a set of immune cells;
generating, by the computer processor, a set of coordinates corresponding to the set of cells;
generating, by the computer processor from the set of coordinates, a first intensity plot corresponding to the set of non-immune cells and a second intensity plot corresponding to the set of immune cells; and
generating, by the computer processor from the first intensity plot and the second intensity plot, the additional set of patient data using the spatial organization modeling technique.

3. The computer-implemented method of claim 2, wherein generating the set of coordinates corresponding to the set of cells comprises:
for each cell in the set of cells:
identifying a location of the cell within the set of stained images, and
generating an x-y coordinate of the cell based on the location of the cell within the set of stained images.

4. The computer-implemented method of claim 1, further comprising:
generating the set of patient data including, for each diagnosed disease of the plurality of diseases using a set of coefficient values generated using the spatial organization modeling technique, generating a set of density estimate curves.

5. The computer-implemented method of claim 4, wherein training the machine learning model using the set of patient data comprises, for each pair of diagnosed diseases in the plurality of diseases:
generating a set of statistical measures between a first one of the pair of diagnosed disease and a second one of the pair of diagnosed diseases, the set of statistical measures indicative of a set of differences between a first set of density estimate curves corresponding to the first one of the pair of diagnosed diseases and a second set of density estimate curves corresponding to the second one of the pair of diagnosed diseases.

6. The computer-implemented method of claim 5, wherein generating the set of statistical measures comprises:
generating the set of statistical measures using a regression model or a classification model.

7. The computer-implemented method of claim 1, further comprising:
transmitting, to an electronic device, information indicating the plurality of probabilities of the additional patient having the plurality of diseases, respectively.

8. The computer-implemented method of claim 1, wherein the set of patient data indicates, for each patient of the plurality of patients, whether that patient responded to an immunotherapy treatment, and wherein the method further comprises:
based on the analyzing, outputting, by the machine learning model, an additional probability of whether the additional patient will respond to the immunotherapy treatment.

9. The computer-implemented method of claim 1, wherein the spatial organization modeling technique is a geographically weighted regression (GWR) technique.

10. A system for using machine learning to assess medical information, the system comprising:
a processor;
a memory storing data associated with a machine learning model; and
a non-transitory computer-readable memory interfaced with the processor and the memory, and storing instructions thereon that, when executed by the processor, cause the processor to:
train the machine learning model using a set of patient data associated with a plurality of patients, the set of patient data (i) generated using a spatial organization modeling technique, and (ii) identifying, for each patient of the plurality of patients, a diagnosed disease of a plurality of diseases,
analyze, by the computer processor using the machine learning model, an additional set of patient data associated with an additional patient, the additional set of patient data generated using the spatial organization modeling technique, and
based on the analyzing, output, by the machine learning model, a plurality of probabilities of the additional patient having the plurality of diseases, respectively.

11. The system of claim 10, wherein the instructions, when executed by the processor, further cause the processor to:
access a set of stained images depicting a set of cells associated with the additional patient, the set of cells comprising a set of non-immune cells and a set of immune cells,
generate a set of coordinates corresponding to the set of cells,
generate, from the set of coordinates, a first intensity plot corresponding to the set of non-immune cells and a second intensity plot corresponding to the set of immune cells, and
generate, from the first intensity plot and the second intensity plot, the additional set of patient data using the spatial organization modeling technique.

12. The system of claim 11, wherein to generate the set of coordinates corresponding to the set of cells, the processor is configured to:
for each cell in the set of cells:
identify a location of the cell within the set of stained images, and
generate an x-y coordinate of the cell based on the location of the cell within the set of stained images.

13. The system of claim 10, wherein the instructions, when executed by the processor, further cause the processor to:
generate the set of patient data including, for each diagnosed disease of the plurality of diseases using a set of coefficient values generated using the spatial organization modeling technique, generating a set of density estimate curves.

14. The system of claim 13, wherein to train the machine learning model using the set of patient data, the processor is configured to, for each pair of diagnosed diseases in the plurality of diseases:
generate a set of statistical measures between a first one of the pair of diagnosed disease and a second one of the pair of diagnosed diseases, the set of statistical measures indicative of a set of differences between a first set of density estimate curves corresponding to the first one of the pair of diagnosed diseases and a second set of density estimate curves corresponding to the second one of the pair of diagnosed diseases.

15. The system of claim 14, wherein to generate the set of statistical measures, the processor is configured to:
generate the set of statistical measures using a regression model or a classification model.

16. The system of claim 10, wherein the instructions, when executed by the processor, further cause the processor to:
transmit, to an electronic device, information indicating the plurality of probabilities of the additional patient having the plurality of diseases, respectively.

17. The system of claim 10, wherein the set of patient data indicates, for each patient of the plurality of patients, whether that patient responded to an immunotherapy treatment, and wherein the instructions, when executed by the processor, further cause the processor to:
based on the analyzing, output, by the machine learning model, an additional probability of whether the additional patient will respond to the immunotherapy treatment.

18. The system of claim 10, wherein the spatial organization modeling technique is a geographically weighted regression (GWR) technique.

19. A computer-implemented method of using machine learning to predict patient response to a treatment for a disease, the computer-implemented method comprising:
training, by a computer processor, a machine learning model using a set of patient data associated with a plurality of patients each being diagnosed with the disease, the set of patient data (i) generated using a spatial organization modeling technique, and (ii) indicating, for each patient of the plurality of patients, whether that patient responded to the treatment;
storing the machine learning model in memory;
analyzing, by the computer processor using the machine learning model, an additional set of patient data associated with an additional patient, the additional set of patient data generated using the spatial organization modeling technique; and
based on the analyzing, outputting, by the machine learning model, a probability of whether the additional patient will respond to the treatment.

20. The computer-implemented method of claim 19, further comprising:
accessing a set of stained images depicting a set of cells associated with the additional patient, the set of cells comprising a first set of cells and a second set of cells;
generating, by the computer processor, a set of coordinates corresponding to the set of cells;
generating, by the computer processor from the set of coordinates, a first intensity plot corresponding to the first set of cells and a second intensity plot corresponding to the second set of cells; and
generating, by the computer processor from the first intensity plot and the second intensity plot, the additional set of patient data using the spatial organization modeling technique.

21. The computer-implemented method of claim 19, further comprising:
transmitting, to an electronic device, information indicating the probability of whether the additional patient will respond to the treatment.

22. The computer-implemented method of claim 19, wherein the set of patient data indicates at least two cell types, and wherein the method further comprises:
generating, by the computer processor using the spatial organization modeling technique, the set of patient data from at least two intensity plots respectively corresponding to the at least two cell types.

23. The computer-implemented method of claim 19, wherein the spatial organization modeling technique is a geographically weighted regression (GWR) technique.

24. The computer-implemented method of claim 19, wherein the treatment is immunotherapy treatment.
